(19) United States
(12) Patent Application Publication
(10) Pub. No.: US 2004/0071779 A1
(43) Pub. Date: Apr. 15, 2004

(54) Gelled laxative compositions

(76) Inventors: Dale Arthur Keiser, Wickenburg, AZ (US); Richard Harry Turner, Mercer Island, WA (US)

Correspondence Address: DALE A. KEISER, 35600 S. ANTELOPE CREEK ROAD, WICKENBURG, AZ, 85390, US

(21) Appl. No.: 10/679,148

(22) Filed: Oct. 3, 2003

Related U.S. Application Data
(60) Provisional application No. 60/417,328, filed on Oct. 9, 2002.

(51) Int. Cl. A61K 9/14

(52) U.S. Cl. 424/488

(57) ABSTRACT

Orally administered gelled formulations to be active in the digestive tract. More specifically, orally administered colonic purgative compositions in gelled form having pleasant odor, taste, and texture.

This application claims the benefit of Provisional Patent Application Number 60/417,328 filed Oct. 09, 2002.

### FIELD OF INVENTION

This invention relates to orally administered gelled formulations to be active in the digestive tract. More specifically, this invention relates to orally administered gelled purgative formulation compositions that may be administered for preparing the colon for surgical or diagnostic procedures or childbirth.

### BACKGROUND

In order to carry out a number of medical procedures, such as colonoscopy, radiographic examination, and childbirth, and in preparation for patients undergoing bowel surgery, it is often critical that the colon be emptied as completely as possible.

A number of orally administered liquid pharmaceutical compositions have been developed for use as gastrointestinal washes for diagnostic purposes or for use as cathartic laxatives. Such preparations consist of aqueous solutions of polyethylene glycol and electrolytes such as sodium sulfate, sodium bicarbonate, sodium chloride and potassium chloride. These orally administered compositions are particularly useful in the rapid washing of the colon for diagnostic purposes. For example, when a powerful gastrointestinal wash is required, such preparations are generally administered in a quantity of about four liters, the composition being typically formulated according to the following: polyethylene glycol 59 g., sodium sulfate 5.68 g., sodium bicarbonate 1.69 g., sodium chloride 1.46 g., potassium chloride 0.745 g. and water to make up one liter. Laxation and relatively thorough evacuation is often significantly improved over enema formulations, and generally without the problems often encountered with enema administrations.

The advantages of using these preparations over other orally administered preparations are a drastic reduction in wash time (from 3-2 days to 4-5 hours) and the minimization of water and electrolyte losses. The advantages that these types of solutions provide are derived from two essential characteristics of the preparation, namely, its isoosmoticity with the physiological liquids, and the balance of the ion species in solution, so as to compensate the transport mechanisms which regulate gastrointestinal absorption. These characteristics result in substantial isotonicity between the preparation and the intracellular and extracellular fluids at the tissues of the digestive tube walls.

Commercially available product embodying these formulations typically utilize a polyethylene glycol formula serving as a nonabsorbable osmotic agent with a mixture of electrolytes for replenishment, so that patients do not become dehydrated. Patients are required to ingest a significant amount of volume for purgation that may include one eight-ounce glass every ten minutes for a total of one gallon of fluid. Due to the fact that the volume is so high, use of this type of formulation is frequently associated with a tremendous amount of distention and significant amounts of nausea. Another serious drawback of these known preparations is their decidedly unpleasant, bitter, and noticeably saline taste which in the more sensitive patients can lead to vomiting thereby preventing ingestion.

In an attempt to avoid the problems associated with the high volume types of preparations, other investigators have utilized ingestible preparations that consist of aqueous solutions of phosphate salts. The aqueous phosphate salt solution produces a tremendous osmotic effect on the intra-luminal contents of the bowel and therefore, evacuation of the bowel occurs with a tremendous increase in the influx of water and electrolytes into the colon. This has been developed for the express purpose of decreasing the volume required in colonic purgations. One such preparation manufactured by Fleet under the brand name Fleet PHOSPHO-SODA™ is manufactured according to the National Formulary monograph for Sodium Phosphates oral solution. This product, as described in the National Formulary (USP 23/NF18, p. 1430), contains dibasic sodium phosphate and monobasic sodium phosphate or phosphoric acid in water. Patients are typically required to take two 1.5 ounce dosages of this preparation, separated by a three to 12 hour interval for a total of three ounces, which is a significant reduction compared to the 128 ounces required by other high volume preparations. Gastroenterologists report excellent cleaning results with the concentrated aqueous phosphate solution.

The major shortcoming of such concentrated aqueous phosphate solution administration is however, that the aqueous solution is extremely unpalatable, so much so that the recommended dosage form is administered ice cold so as to minimize the objectionable saline taste. Often patients complain of severe nausea and vomiting, possibly secondary to the extremely salty taste of the preparation. Frequently, patients cannot even tolerate the ingestion of this preparation at the initial dose and often the second dose becomes even more problematic due to the unpalatable extremely salty taste, even when the taste is partially masked by the use of flavoring agents.

An additional shortcoming of such concentrated aqueous phosphate solution administration is the occurrence of side effects related to exposure of the intestinal lining to a rapid increase in phosphate salt concentration. Side effects include cramping, nausea, and vomiting.

Thus, while concentrated purgation solutions represent an improvement over other methods of inducing purgation, the unpalatable taste and the unpleasant side effects are serious shortcomings.

Other investigators have utilized capsules and tablets to contain and deliver dry formulations as a solution to the problem of unpalatable taste. (See U.S. Pat. No. 5,616,346 to Aronchick Apr. 1, 1997, and U.S. Pat. No. 5,997,906 to Wood, Dec. 7, 1999.) Gastroenterologists have reported reduced fecal cleansing and a problem with increased foam in the upper colon with these solid forms as compared to the aqueous forms, most likely a consequence of the use of binders and coatings in the formulations. The administration of these nonaqueous formulations typically requires that 3 tablets be taken at a time with 8 ounces of clear liquid every 15 minutes for a total of 20 tablets. Then, 3-5 hours before the medical procedure, the process is repeated with another 20 tablets for a grand total of 40 tablets and 112 ounces of liquid. This regimen is quite demanding for a patient.

From the foregoing, it can be seen that the problem with unpalatable taste and unpleasant side effects is still a serious problem with known preparations. Attempts to resolve the problem of unpalatable taste through the use of capsules or tablets to deliver a dry formulation have introduced complexity for the patient and have proven to be less effective than the aqueous formulations.

### SUMMARY

Accordingly, a gel carrier component:

(1) temporarily retains an unpleasant tasting laxative component so as to mask the unpleasant taste while preserving the efficacy of the laxative component.

(2) provides for the slowed release of a high strength laxative component in the intestine so as to minimize side effects such as cramping and nausea that could occur with the otherwise rapid introduction of the laxative component.

### DETAILED DESCRIPTION

The embodiments of the invention described here provide a laxative composition having a pleasant odor and taste and having the effect of reduced side effects compared to known formulations. These embodiments relate to a colonic purgative formulation that comprises a carrier component in gelled form, and a laxative component. In the first embodiment, the carrier component comprises gelatin, a flavoring, a sweetener, a dye, and a preservative, and the laxative component comprises dibasic sodium phosphate and monobasic sodium phosphate according to the National Formulary monograph for Sodium Phosphates oral solution (USP 23/NF18, p. 1430). This embodiment may be realized in several ways including that detailed in the following example:

### EXAMPLE 1

This example formulation comprises a gelatin desert, for example, JELL-O™ brand desert mix and a laxative, for example Fleet brand PHOSPHO-SODA™. This combination provides all of the elements of the preferred embodiment. The desert mix provides gelatin, flavoring, dye, sweetener, stabilizer, and preservative. The exemplary PHOSPHO-SODA™ provides the appropriate ratio of dibasic sodium phosphate and monobasic sodium phosphate (according to the National Formulary monograph for Sodium Phosphates oral solution (USP 23/NF18, p. 1430)) plus additional stabilizers, preservatives, and flavoring.

Combine three ounces of the exemplary orange flavored JELL-O™ brand desert mix with 130 ml. of water. Heat to near boiling while stirring until solids are dissolved.

In a separate container, combine 65 ml. of water with 45 ml. of Fleet brand PHOSPHO-SODA™. Heat to near boiling.

Slowly combine the diluted purgative into the gelatin mixture while stirring.

Slowly cool the mixture in an 8-oz. container to about 35 degrees Fahrenheit.

This example provides an 8-ounce serving of gel containing an effective 1.5-ounce purgative dose.

In a second embodiment, the carrier component comprises gelatin, a flavoring, a sweetener, a dye, and a preservative, and the laxative component is magnesium citrate. This embodiment may be realized in several ways including that detailed in the following example:

### EXAMPLE 2

This example formulation comprises a Gelatin (for example, Gelatin, Type A, 25 Bloom, 50 mesh, from Great Lakes Gelatin, PO Box 917, Grayslake, Ill. 60030.) and a laxative, for example, Long's Drug Co. brand Magnesium Citrate Oral Solution™. This combination provides all of the elements of the second preferred embodiment wherein the Long's Magnesium Citrate Oral Solution™ provides flavoring, dye, sweetener, stabilizer, preservative and the laxative component, magnesium citrate. The gelatin provides the gelling component.

Transfer 10 fl oz of the laxative into a container. Add 8.75 gm of gelatin on top of the solution. Stir until the gelatin is dispersed. Wait until the gelatin has hydrated, about 15 minutes. Heat to near boiling while stirring until all solids are dissolved.

Slowly cool the mixture to about 35 degrees Fahrenheit. This example provides a 10-ounce serving of gel containing an effective dose of oral magnesium.

In a third embodiment, the gelled carrier component comprises agar, a flavoring, a sweetener, a dye, and a preservative, and the laxative component comprises magnesium citrate. This embodiment may be realized in several ways including that detailed in the following example:

### EXAMPLE 3

This example formulation comprises a gelling agent, Agar (for example, Sigma brand Agar A-7002 Lot 71K0093), a laxative, for example, Fleet brand PHOSPHO-SODA™ and a composition of water, flavoring, dye, sweetener, stabilizer, and preservative, for example, Orange Gatorade™ (The Gatorade Company). This combination provides all of the elements of the third preferred embodiment. The exemplary Orange Gatorade™ provides flavoring, dye, sweetener, stabilizer, and preservative. The exemplary PHOSPHO-SODA™ provides the appropriate ratio of dibasic sodium phosphate and monobasic sodium phosphate [according to the National Formulary monograph for Sodium Phosphates oral solution (USP 23/NF18, p. 1430)] plus additional stabilizers, preservatives, and flavoring. The Agar powder provides the gelling component.

Combine 1.5-ounce of the purgative with 3.5-ounce of the exemplary Gatorade™.

Add 2 gm of Agar to the mixture. Stir the mixture to disperse the Agar.

Heat to near boiling while stirring until the solids are dissolved.

Slowly cool the mixture to about 35 degrees Fahrenheit.

This example provides a 6-ounce serving of gel containing an effective 1.5-ounce purgative dose.

The unpleasant taste of known aqueous purgative formulations is a result of drenching the taste receptors in the patient's mouth with the high concentration of salts present in the purgative formulations. The described embodiments do not change the basic composition of the known formulations, but instead, temporarily retain the formulations within a gel so that the taste receptors in the mouth are minimally exposed to the offending formulation.

In each described embodiment, the carrier component gel retains a pharmaceutically active component. As the patient ingests the gel formulation, the patient tastes only that minute percentage of active component that is exposed at the surface of the gel. Chewing prior to swallowing exposes more gel surface area, yet only a very small percentage of the total active saline component is exposed. The laxative component is released from the gel in the patient's stomach and small intestine as the gel dissolves. With the strong saline taste minimized, the flavoring and sweetener added to the carrier become evident to the patient. The gel formulation has a pleasant taste, is presented in a colorful and familiar form, and is readily accepted by the patient.

In addition to providing greatly improved taste over known formulations, the described embodiments provide the further advantage of reduced side effects over known formulations. Side effects such as cramps and nausea are often associated with the known purgative formulations. These side effects are a result of the shock induced by rapid changes in osmotic strength in the intestine. The rapid change in osmotic strength is the result of the rapid introduction, to the intestine, of the active laxative components of the known formulations. The described embodiments do not change the active laxative components of the formulations, but instead, provide for the slowed release of those components in the intestine. The slowed change in osmotic strength provided by the described embodiments proportionally reduces the shock otherwise associated with a rapid change in osmotic strength.

### ALTERNATIVE EMBODIMENTS

Examples 1, 2, and 3 describe embodiments where the gel is molded into a single mass. The gel may alternately be molded into any of several forms including various diameter balls, discs, strips, or squares. For example, a small ice cube tray may be used to mold several one-half inch cubes. A dose may comprise a number of smaller-than-bite-size gel shapes that are stirred into a glass of water and then drunk without chewing.

The formulation of Example 1 provides a gel with a consistency similar to that of the familiar JELL-O™ brand desert. Many types of gelatins are available, as are many types of each of the other gelling agents listed previously. The type or concentration of gelatin or other gel agent may be adjusted to produce different degrees of firmness of the gel as well as to change other physical properties of the gel such as melting temperature and texture. The properties of the gel carrier component may be selected to optimize the characteristics of an end formulation that may or may not include flavorings, sweeteners, fragrances, dyes, stabilizers, or preservatives.

Each of the previously described embodiments is a gelled formulation that is presented to the patient in a ready-to-use gel form. Alternatively, the formulation can be prepared and presented to the patient in forms other than a ready-to-use gel whereby the patient may prepare the gel form. Several methods of making and supplying the components of the gel formulation are possible including the following:

Method 1) Each of the dry components of the formulation can be supplied separately in a single package whereby the patient may prepare the gel by adding water to the dry components, then heating, mixing, and cooling the mixture.

Method 2) The dry components of the formulation can be supplied pre-mixed in a single package whereby the patient may prepare the gel by adding water to the dry components, then heating and cooling the mixture.

Method 3) The dry components and liquid components of the formulation can be supplied separately in a single package whereby the patient may prepare the gel by appropriately mixing, heating, and cooling the mixture.

Method 4) The formulation may be supplied to the patient in a liquid form such that when the liquid is cooled, it forms a gel.

The compositions described will mask taste while the composition is in its gelled form. The gelled composition will melt, depending on the specific composition, at elevated temperature. In the case of compositions that will melt at a temperature lower than the temperature of a patient's mouth, the method of taking the compositions becomes important. The following methods of taking the compositions will enhance the taste-masking effectiveness of the composition.

Method 1) The gelled composition may be chilled to a temperature in the range of 30 to 37 degrees Fahrenheit. The composition will remain gelled for a longer time if it is colder when taken by the patient.

Method 2) The gelled composition may be taken in small portions, such as portions less than 1 teaspoon, so that each portion may be swallowed after minimal chewing. Ideally, the portion would be swallowed with no chewing.

Method 3) The patient may drink a small amount of cold liquid before, and, or with, and, or after each portion of the composition. This method cools the patients mouth resulting in less heat transfer to the gelled composition and hence, less melting of the gel. This method also rinses away some of the unpleasant tasting component that may have escaped the gel. This method also encourages increased fluid intake as generally prescribed for the bowel cleansing process.

The foregoing descriptions are illustrative of several embodiments. The descriptions are not intended to limit the invention to the specific formulations shown and described, but instead it will be appreciated that adaptations and modifications will become apparent from the present disclosure and are intended to be within the scope of the claims.

We claim:

1) A homogeneous gelled composition comprising an edible gelling component and a laxative component.

2) The composition according to claim 1, further comprising at least one additional component selected from the group consisting of flavors, dyes, fragrances, stabilizers, sweeteners, and preservatives.

3) The composition according to claim 1, wherein said gelling component comprises at least one gelling substance selected from the group consisting of gelatin, pectin, carrageenen, agarose, agar, and agar-agar.

4) The composition according to claim 1, wherein said laxative component comprises at least one ingredient selected from the group consisting of bisacodyl, docusate, polyethylene glycol, sodium sulfate, sodium bicarbonate, sodium chloride, potassium chloride, potassium sulfate, sodium phosphate, phosphoric acid, and magnesium citrate.

5) The composition according to claim 1, wherein said laxative component is a Sodium Phosphate oral solution as described by the National Formulary monograph in the National Formulary (USP 23/NF18, p. 1430).

6) The method of making a gelled laxative formulation whereby the composition is prepared in its entirety at the factory and is supplied in a ready-to-use form.

7) The method of making a gelled laxative formulation whereby components of the composition are supplied to the user and the user further prepares a gelled form for use.

8) The method according to claim 7 whereby dry components are supplied in a mono-package, whereby the user adds water and further prepares a gelled form for use.

9) The method according to claim 7 whereby dry components and liquid components are supplied individually, whereby the user combines the components and further prepares a gelled form for use.

10) The method according to claim 7 whereby dry components and liquid components are supplied individually, whereby the user combines dry and liquid components with water and further prepares a gelled form for use.

11) The method of using a gelled laxative formulation whereby the gelled composition is administered in bite-size pieces.

12) The method according to claim 11 wherein the gel is cold.

13) The method according to claim 11 whereby the gelled composition is swallowed after minimal chewing.

14) The method according to claim 11 whereby the administration of the gelled composition is preceded by and, or accompanied by, and, or followed by, a drink of cold liquid.
